(19) United States
(12) Patent Application Publication
(10) Pub. No.: US 2005/0203402 A1
(43) Pub. Date: Sep. 15, 2005

(54) Digital ultrasound beam former with flexible channel and frequency range reconfiguration

(76) Inventors: Bjorn A.J. Angelsen, Trondheim (NO); Tonni F. Johansen, Trondheim (NO)

Correspondence Address: Lance J. Lieberman, Esq.;Cohen, Pontani, Lieberman & Pavane, Suite 1210, 551 Fifth Avenue, New York, NY, 10176, US

(21) Appl. No.: 11/054,399

(22) Filed: Feb. 9, 2005

Related U.S. Application Data
(60) Provisional application No. 60/543,241, filed on Feb. 9, 2004.

(51) Int. Cl. A61B 8/02; A61B 8/06; A61B 8/12

(52) U.S. Cl. 600/447

(57) ABSTRACT

A digital ultrasound beam former for ultrasound imaging, that can be configured by a control processor to process the signals from ultrasound transducer arrays with variable number of elements at variable sampling frequencies, where the lowest sampling frequency allows for the highest number of array elements. The maximal number of array elements is reduced in the inverse proportion to the sampling frequency. Parallel coupling of transmit/receive circuits for each element allow adaption of the receive Noise Figure and transmit drive capabilities to variations in the electrical impedance of the array elements.

### RELATED APPLICATIONS

This application claims priority from U.S. Provisional Patent Application Ser. No. 60/543,241 which was filed on Feb. 9, 2004.

### BACKGROUND OF THE INVENTION

1. Field of the Invention

The present invention is directed to methods and instrumentation of ultrasound imaging in a wide frequency range where the digital beamformer is reconfigurable in terms of number of channels versus frequency range.

2. Description of the Related Art

Digital ultrasound beam formers for medical ultrasound imaging have the last decade become feasible due to improved functionality of analog to digital converters (ADCs) and digital integrated circuit technology. However, the requirements on the beam former in terms of number of channels, frequency bandwidth, signal dynamic range, etc., highly depend on the application and the resolution versus depth penetration required.

The cost of the beam former per channel is dominated by the cost of the ADCs, which increases with number of bits and highest sampling frequency of the ADC. The requirement for number of bits is determined by the required dynamic range where blood velocity imaging in the heart puts the strongest requirement on the dynamic range (and number of bits) due to the demanding filtering of the wall signals to retrieve the blood signal for the velocity processing. Non-cardiac imaging requires less dynamic range and number of bits in the ADCs, and an increase in the center frequency and the bandwidth further reduces the dynamic range in the signal and hence the required number of bits. Reducing the transducer array element dimensions also reduces the number of required bits per channel.

It is hence a need for a beam former where the number of channels, dynamic range, and frequency range can be reconfigured for the particular application at hand.

The largest number of channels are found with the phased arrays, where the element pitch is λ/2, where λ=c/f is the wave length of ultrasound in the tissue with ultrasound propagation velocity c (˜1.54 mm/μsec) and f is the ultrasound frequency. With switched linear or curvilinear arrays, the element pitch can be increased to λ−1.5λ, increasing the aperture by a factor 2-3 compared to the phased array with the same number of elements, or with limited increase in the aperture allows for a reduction in the number of electronic channels in the beam former. With the beam axis along the surface normal of the array (no angular direction steering of the beam), one can also do analog summation of the signals for the pair of elements with symmetric location around the aperture center, hence reducing the required number of ADCs by a factor 2.

The annular arrays require even less number of delay channels. As the element areas are larger than for the switched arrays, their electrical impedance is proportionally less, and it is practical to parallel couple analog channels for each element of the annular array so that for similar apertures and frequencies one gets about the same number of analog channels for the annular and the switched arrays. This statement specially applies to the annular array design described in U.S. Pat. No. 6,622,562 Sep. 23, 2003, where the outer elements have specially large area.

Manufacturing technology gives a limitation on the lowest pitch of the array elements, where λ/2 pitches are achievable for frequencies up to 10 MHz with current transducer array technology. This is hence the highest frequency where the phased array method has been used, while for higher frequencies one is using switched arrays where the lowest manufacturing pitch with current technology allows frequencies up to 20-30 MHz. Current experimental manufacturing techniques allow frequencies of switched arrays up to ˜50 MHz.

The annular arrays have the fewest number and hence the largest elements for a given aperture. They therefore allow the use of the highest frequencies, even up to 100 MHz with current technology. One should also note that the phased array image is mainly interesting for imaging between ribs and from localized areas, where a highest frequency of 10 MHz is adequate, while the image formats of the switched and annular arrays are applicable over the whole frequency range. With some intraluminal catheter and surgical applications one can see the sector image format of the phased array also being attractive for frequencies above 10 MHz. With new transducer technology based on ceramic films or micromachining of silicon (cmut—capacitive micromachined ultrasound transducers), one sees opportunities for manufacturing of phased arrays with center frequencies above 10 MHz.

It is hence a need for a beam former that can run a large number of channels for wide aperture phased and linear arrays up to a center frequency f₀˜15 MHz, with a less number of channels for frequencies up to 2f₀˜30 MHz with switched arrays and annular arrays, and an even less number of channels for frequencies up to 4f₀˜60 MHz to be operated with switched and annular arrays.

### SUMMARY OF THE INVENTION

The present invention gives a solution to this need, where the digital beam forming is done with field programmable digital circuits that are programmed by a central processor, like a PC, that provides a reconfigurable front end for different sampling rates and number of channels depending on the type of array and frequency range that is used. The digital circuits can either be Application Specific Integrated Circuits (ASICs) that are designed to be field programmable, or Field Programmable Gate Arrays (FPGAs).

The essence of the invention is that a number of N analog to digital converters (ADCs) are operated at a sampling frequency fₛ, usually close to their maximum sampling frequency for cost reasons, and are connected at their input to an analog multiplexer that allows the ADC to take input from several, selectable analog beam former channels, and the output of each ADC is connected to one or more field programmable digital beam forming circuits. When lower sampling frequencies are allowed for the signal bandwidths that are used, each ADC can through selectable activation of the input mux, serve several analog beam former channels with a reduced sampling frequency fₛ/L, where L is the number of analog beam former channels that are digitized by the same ADC. This allows L*N number of analog channels to be processed at the lower sampling rate fₛ/L per channel.

At a higher bandwidth, each ADC can convert one analog channel at the sampling frequency fₛ. At even higher bandwidths groups of several ADCs in each group can via the input mux be connected to each transducer element with a phase difference of the sampling frequency within each group of ADCs, so that the effective sampling frequency of each element signal is Mfₛ, where M is the number of ADC that digitizes each analog channel.

The digital dynamic range can be increased with lower signal bandwidths by using increased sampling rates related to the bandwidth (over sampling), followed by digital low pass filtering of the signals that increases the number of bits and reduces the sampling rate.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

In the drawings:

FIG. 1, shows a front end embodiment according to the invention where a front end is configured to a lowest sampling frequency allowing a highest number of transducer elements;

FIG. 2, shows other configurations of the front end in FIG. 1, that provides other sampling frequencies with other number of elements; and

FIG. 3 shows yet another example embodiment of a front end according to the invention with four different configurations with different sampling frequencies and maximal number of elements.

### DETAILED DESCRIPTION OF THE PRESENTLY PREFERRED EMBODIMENTS

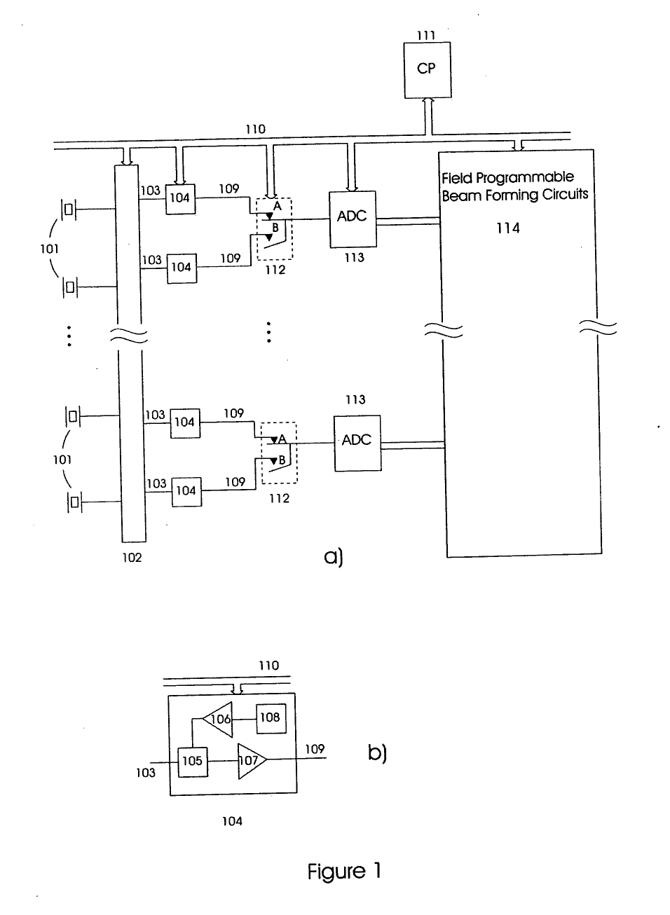

FIG. 1 illustrates one embodiment in the general spirit of the invention, where 101 indicates elements in an ultrasound transducer array, where each element is connected to an array coupling means 102, that provides selectable connection of the array elements to the inputs 103 of sets 104 of T/R (transmit/receive) circuits.

Essential elements of the T/R circuits are shown in FIG. 1b, where the input 103 connects to a transmit/receive switch 105, that connects the transducer array to transmit amplifiers 106 during the pulse transmit period, and to receiver amplifiers 107 during the receive period. The transmit amplifiers are driven from signal generators 108 that are set up via the bus 110 by the control processor 111. The transmit pulse can be triggered by a signal on the bus or through other means. The generator for example provides a delayed pulse transmit, where the delay is set for adequate focusing and direction steering of the transmit beam. The delay can also include corrections for wave front aberrations in heterogeneous tissue, and the generator can also include amplitude corrections for the same aberrations.

The output of the receiver amplifier 109 is fed further to one of the inputs of many-to-one multiplexers 112, whose outputs are fed to inputs of analog to digital converters (ADCs) 113. The ADCs are sampling and converting to digital form their analog inputs at a sampling rate fₛ, which in some embodiments could be controlled by the processor 111 through the bus 110. The output of the ADCs are fed to digital beam forming circuits 114 that can be programmed by the control processor 111 as described below.

The array coupling means 102 connects selected elements to selected sets of J T/R circuits, where the minimum value of J is one as illustrated in FIG. 1a. In this set-up, the mux's 112 are set up to for each 2^{nd} sample to connect the upper and lower (A and B) analog channels to the ADCs. The ADC outputs are synchronously separated into the two element signals in the beam forming circuits 114. Hence, each ADC is converting L=2 element signals, each with a sampling frequency fₛ/L.

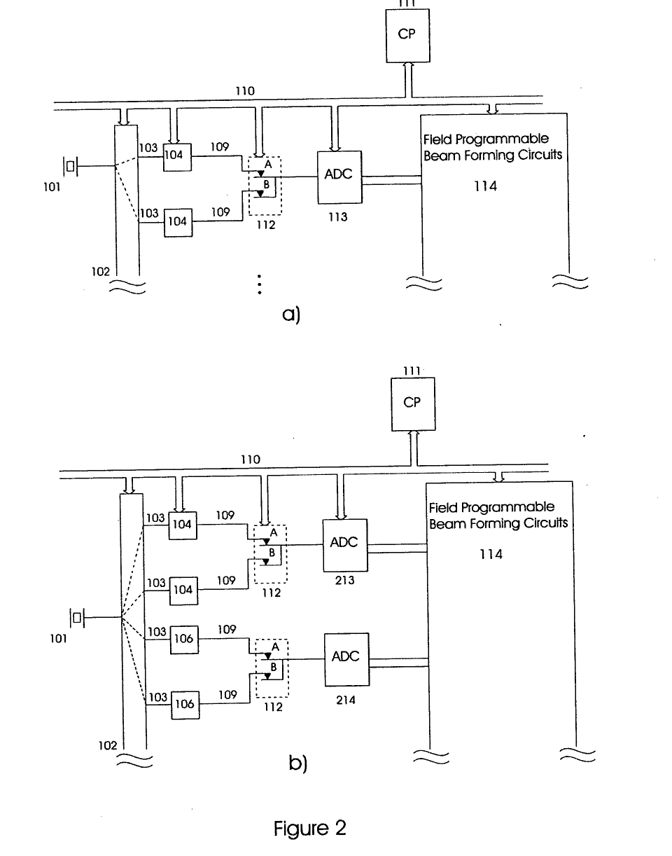

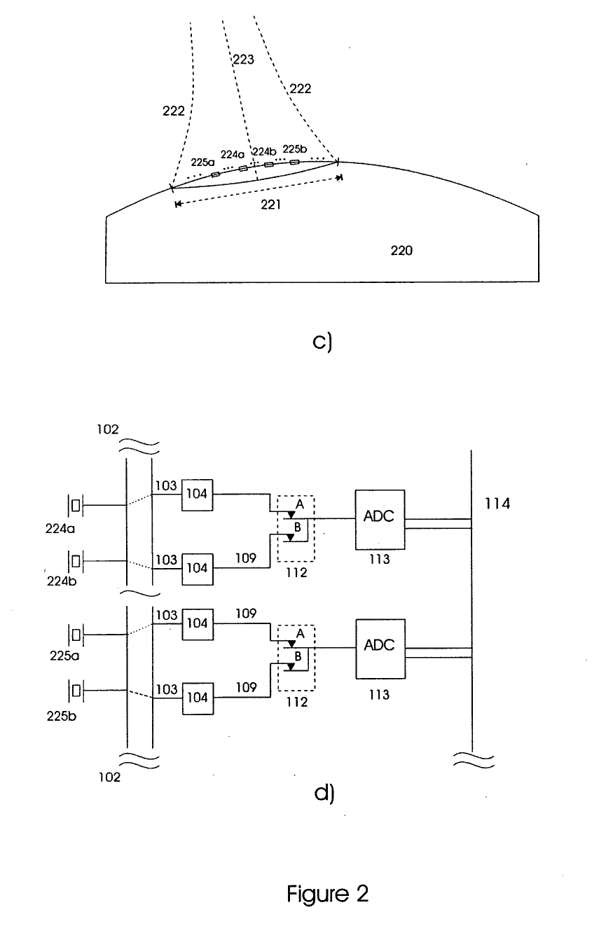

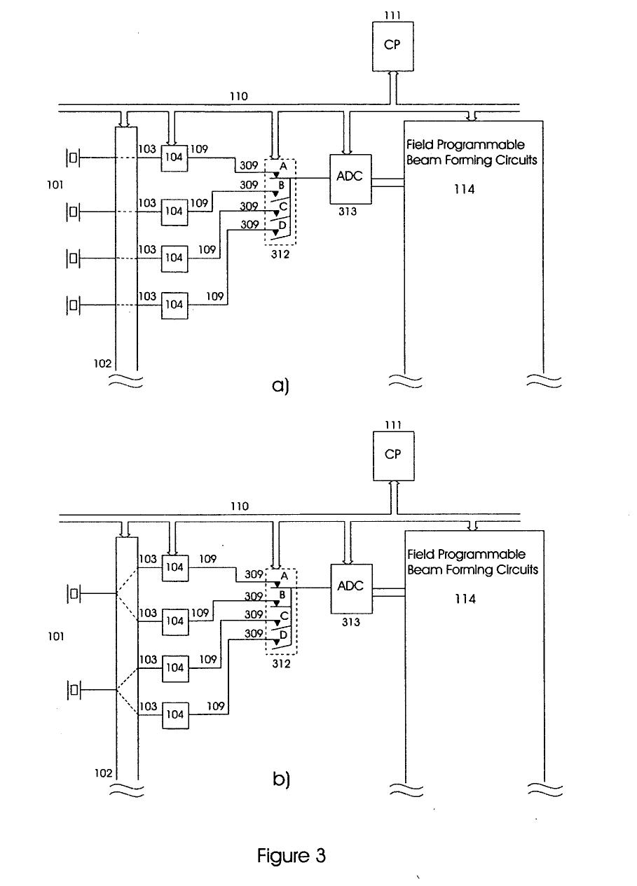

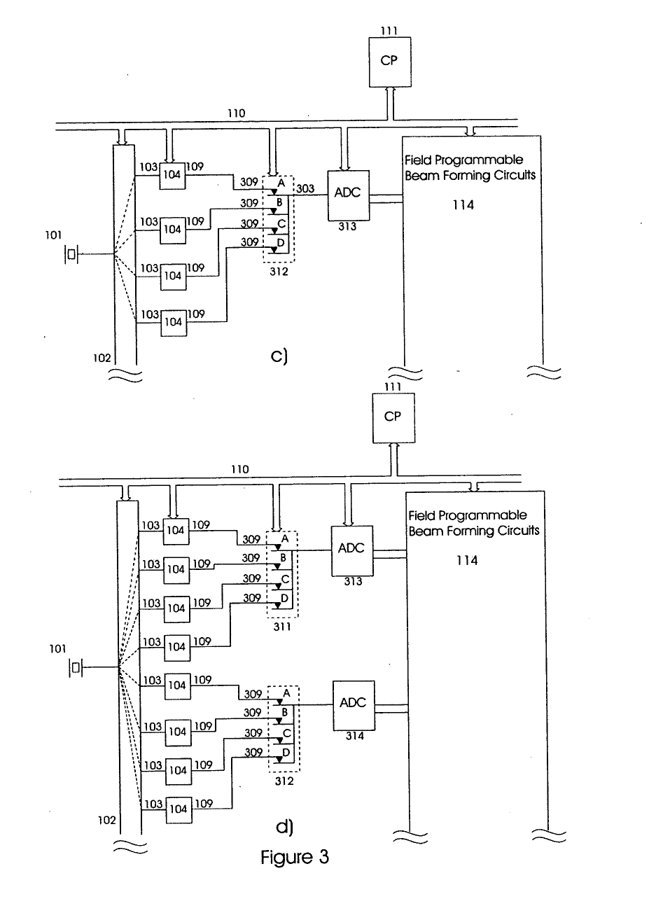

Other values of J are shown in FIGS. 2 and 3, where FIGS. 2a and 2b show connections with J=2 and 4, respectively, and FIG. 3a to 3d show connections with J=1, 2, 4, 8 respectively. The connection selection can in its simplest form be done in a connector that couples the transducer array to the ultrasound beam former, where the group of array transmit/receiver circuits that connects to each array element is hardwired in the connector. The actual coupling to the transmit/receive circuits is stored by a code in the connector that in this embodiment can be read by the control processor 111 over the bus 110 so that the control processor has information about the array to T/R circuit connections for a particular probe. In a more flexible embodiment, the array coupling means 102 can contain flexible multiplexers that are set up by the control processor 111 over the bus 110, so that one can have selectable element to T/R circuit connections for one particular transducer array, in a manner known to anyone skilled in the art.

In conjunction with the various couplings between the transducer array and the T/R circuits, the ADC multiplexers are set up for matched functioning as illustrated in FIGS. 2 and 3. FIG. 2a shows a situation where each transducer element is connected to two T/R circuits (J=2) and the ADC multiplexers 112 are programmed so that both switches A and B are connected. The signal from each element is then sampled and AD converted at the sample rate fₛ of the ADCs. FIG. 2b shows yet another configuration of the front end, where each array element is coupled to four T/R circuits (J=4). Both of the shown AD converters, 213 and 214, are then receiving the same element signals at their inputs which are sampled and AD converted at the rate fₛ. The sampling time points of ADC 214 are delayed ½fₛ in relation to those of the ADC 213, and the signals from the two ADCs are merged in the beam forming circuits 114 into one digital signal for the selected element with sampling rate Mfₛ, where in this particular configuration M=2.

The digital beam forming circuits 114 are programmable to adapt to the different configurations in FIGS. 1 and 2. In the operation indicated in FIG. 1a, the outputs of each ADC is separated into the 2 element signals in the digital beam former circuits 114, with the sampling frequency fₛ/2 per element signal. In this configuration the beam former can handle a phased array with 2N elements with angular direction steering of the beam. The beam former can in this configuration also handle a switched array with aperture of 2N elements and angular direction steering of the beam.

With the operation indicated in FIGS. 1a and 2a,b, the digitized signals represents individual element signals, which could handle phased and switched arrays with angular direction steering of the beam for L*N or N/M elements. For a switched array without angular direction steering of the beam, one can add the analog signals of the pair of elements that have symmetrical location around the aperture center, before they are digitized by the same ADC. This allows the beam former to operate switched array apertures without angular direction steering of the beam with twice as many elements as with direction steering of the beam. In the following we refer to apertures without direction steering of the beam as symmetric delay apertures, and with direction steering of the beam as asymmetric delay apertures.

This is illustrated in FIG. 2c, where 220 shows a side view of a slightly curved array where a group 221 of elements have been selected for an active aperture to produce an ultrasound beam indicated with the lines 222 and the beam center axis 223. The elements have a pair-wise symmetric positioning around the aperture center, indicated by the example element pairs 224a,b and 225a,b. The array coupling means 102 is in this example a multiplexer or cross-point switch that connects symmetric pairs of elements to the T/R circuits that are connected to the same ADC multiplexer, as illustrated in FIG. 2d. The multiplexers 112 are in this example designed together with the receiver amplifier outputs so that their output produces the sum of the (current sum or voltage sum) pair element signals as inputs to the ADCs 113 that provides the digitized sum signal to the digital beam forming circuits 114, where the signals are appropriately delayed, amplitude scaled and summed to form a dynamically focused beam with beam central axis 223 normal to the array surface. In a modified embodiment, the signals from the paired elements around the beam axis, can be summed before the T/R circuits by multiplexers in the array coupling means, and the T/R circuits, multiplexers, ADCs, and beam forming circuits operating as each sum of symmetric element signals was a single signal.

In the configuration of the beam former shown in FIG. 2b, the output of two paired ADCs are merged into one element signal in the beam former circuits 114 to give a sampling frequency of the element signal of 2fₛ. With implementation of the digital beam former in Application Specific Integrated Circuits (ASICs), this programmability can be taken care of in the ASIC design, so that the different operations are selectable by the system processor. Highly interesting are also Field Programmable Gate Array (FPGA) circuits, where the programs for the different operations are loaded over the bus 110 from the control processor 111.

By example, with ADCs operating at fₛ=100 MHz, the setup indicated in FIG. 1 gives a beam former sampling frequency of fₛ/2=50 MHz (L=2) with a highest ultrasound center frequency ˜15 MHz operating asymmetric delay apertures of 2N transducer elements. The configuration in FIG. 2a gives a sampling frequency of fₛ=100 MHz (L=1) with a highest ultrasound center frequency of ˜30 MHz operating an asymmetric delay aperture of N transducer elements and a symmetric delay aperture of 2N elements. The configuration in FIG. 2d gives a sampling frequency of fₛ=100 MHz (M=1) with a highest ultrasound center frequency of ˜30 MHz and operates switched arrays with asymmetric delay apertures of N elements and symmetric delay apertures of 2N elements. The configuration in FIG. 2b gives a beam former sampling frequency of 2fₛ=200 MHz (M=2) with a highest ultrasound center frequency of −60 MHz operating N/2 transducer elements with asymmetric delay apertures, and N transducer elements with symmetric delay apertures.

One should also note that increase in the digital signal dynamic range can be obtained for low signal bandwidths by using a higher than necessary sampling frequency, and reducing the sampling frequency through digital low pass filtering. Hence, for an N element array with asymmetric delay aperture with so low signal bandwidth that fₛ/2 is an adequate sampling frequency, one can sample at fₛ and through low pass filtering reduce sampling frequency to fₛ/2 with an increase in the effective dynamic range of the digital signal by the square root of 2. Similarly, for an array of N/2 elements with asymmetric delay aperture and bandwidth of fₛ/2, one can sample the signal at 2fₛ and through low pass filtering reduce the sampling frequency to fₛ/2 with an increase in the digital signal dynamic range of 2. With symmetric delay apertures one can do the same with 2N and N elements.

FIG. 3 shows yet another example embodiment according to the invention, where the multiplexers 312 now connects 4 inputs 309 to one output. The ADCs 313 and 314 converts their analog inputs at a sample rate fₛ. In the configuration of FIG. 3a, each array element 101 is connected to a single T/R circuit (J=1) so that each element signal connects to only one multiplexer input. The switches A, B, C, and D are connected in a sequence, so that the ADC 313 in a sequence is sampling and AD converting each of the 4 element signals of the connected multiplexer with a sampling rate fₛ/L where L=4. In the configuration of FIG. 3b, each array element 101 is connected to 2 T/R circuits (J=2). The switches (A,B) and (C, D) are connected in parallel in a sequence, so that the element signals are each sampled at a rate fₛ/L where L=2. In the configuration of FIG. 3c, each array element 101 is now connected to 4 T/R circuits (J=4), and all switches A, B, C, D are connected in parallel so that each element signal is sampled at the rate fₛ/L with L=1.

FIG. 3d shows a configuration of the front end with 4-to-1 multiplexers, where each array element 101 is connected to 8 T/R circuits (J=8), and all the switches A, B, C, D are connected in parallel. All the switches A, B, C, and D of the multiplexers 311 and 312 are connected so that both ADCs 313 and 314 are sampling the same element signal at a rate fₛ. The sampling time points of ADC 314 are delayed ½fₛ in relation to those of ADC 313, and the signals from the two ADCs are merged in the beam forming circuits 114 into one digital signal for the selected element with sampling rate Mfₛ, where in this particular configuration M=2.

With no angular direction steering of the beam, one can for the 4-to-1 multiplexers in FIG. 3 set up the array coupling means for symmetric delays around the aperture center similar to FIGS. 2c and 2d, so that the beam former operates switched array symmetric delay apertures with twice the number of elements as with asymmetric delay apertures. The array coupling means is for this operation multiplexers that connects the array elements to the appropriate T/R circuits to obtain the same delay for pairs of array elements that are symmetric around the aperture center.

In the example configurations of FIGS. 1-3, the array elements are coupled to a single T/R circuit for the lowest sampling frequency fₛ/L (L=2 in FIG. 1 and L=4 in FIG. 3a), while with increasing sampling frequencies an increasing number of T/R circuits are coupled in parallel to each element. This parallel coupling is in most situations advantageous as the increasing sampling rate is used with increasing center frequency of the array elements, and the electrical element impedance is most often reduced with increasing center frequency. The Noise Figure of the receiver is then improved by coupling more receiver amplifiers in parallel for each element, and the parallel coupling of transmit amplifiers provides improved transmit drive capability of the array elements.

If for some reason, the area or the material of the array elements are varied so that the electrical impedance of the array elements has limited or no drop with increase in center frequency, one can set up the array coupling means 102 and the multiplexers so that adequate sampling frequency is obtained with less T/R circuits coupled to each element, in a manner that is clear to anyone skilled in the art, based on the disclosures so far. For example, one could in FIGS. 2a and b set up the array coupling means and the multiplexers so that only the upper T/R circuit is used where the switch A is closed and switch B is open all the time. Similarly, in FIG. 3b one could couple either one or two (J=1 or 2) T/R circuits to each array element, while in FIG. 3c one could select between J=1, 2, 3, and 4 T/R circuits coupled to each element. In FIG. 3d one could similarly select between J=2, 4, 6, and 8 T/R circuits coupled to each array element, and still be able to obtain a sample frequency of each array element signal of 2*fₛ by merging the outputs of the ADCs 313 and 314 into one element signal in the beam forming circuits. The front end can hence not only be configured for variable sampling frequency in relation to the center frequency of the actual array, but also to variable electrical element impedance so that best Noise Figure of the receiver and drive capabilities of the transmitter is achieved.

With annular arrays, one has the fewest number of elements for a given area of the aperture, and hence also the lowest electrical element impedance for each element. For best Noise Figure of the receiver and also drive capabilities of the transmitter, one can then conveniently couple a larger number of T/R circuits to the same element, where a larger number M of ADCs are sampling each element signal with a time delay between the samples of each ADC of 1/Mfₛ. The signal outputs of all the M ADCs sampling one element signal are then merged in the beam forming circuits to represent the signal from this particular element sampled at a rate Mfₛ. As the annular array has the largest and fewest elements for a given aperture, the front end can hence be configured to the highest sampling rate for the annular arrays. A particular design of an annular array is given in U.S. Pat. No. 6,622,562, where the outer elements have wider area, and hence lower electrical impedance, than the inner elements. The number of T/R circuits coupled to each element should then be proportional to the element area, which means that the area of the outer elements should be selected as a rational number times the area of the inner elements, so that each T/R circuit handles the same element area, and hence also electrical impedance, for all elements.

The example embodiments above hence illustrates a basic principle of a digital beam former that is configured by a processor to operate with different sampling frequencies and number of transducer elements, the beam former making optimal use of the ADCs for highest possible number of transducer elements at a given ultrasound frequency, and being able to adapt the sampling frequency to higher ultrasound frequencies where less number of transducer elements are needed for the beam forming, and the transmit/receive circuits are parallel coupled to adapt to the reduced impedance of the higher frequency transducer elements. Essential in this configurability is the use of field programmable digital beam forming circuits, implemented as field programmable ASICs or FPGAs, where the beam forming circuits are programmed for each particular array element to ADC configuration.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

1. A computer configurable digital ultrasound beam-former for steering the direction and/or the focus of an ultrasound beam from ultrasound transducer arrays of different types with variable number of elements and frequencies, said beam-former comprising:
K sets of analog transmit/receive circuits, each set containing a transmit amplifier and a receiver amplifier, K being a whole number, and
an array coupling means that can couple signals to and from said array elements or groups of array elements to inputs of groups of transmit/receive circuits for example through hardwiring in the connector for each individual array or through selectable electronic switches, and
N analog multiplexers that selectably connects outputs or sums of outputs of said receiver amplifiers to a single output, N being a whole number less or equal to K, and
N analog to digital converters (ADCs) operating at a conversion rate fₛ, and where the input of each ADC is connected to the output of said multiplexers in a one-to-one connection, and
one or more field programmable digital beam forming circuits to which the outputs of said ADCs are coupled as inputs, said digital beam forming circuits being able to sort the outputs of said ADCs into digital samples of received signals from said elements or groups of elements, introducing delay and amplitude modifications of said sorted signals and combining them into one or more beam signals, and
a functional control processor at least enabled to selectably configure the functional operation of the beam former through functional interaction with said array coupling means, said multiplexers, and said beam-forming circuits, selectably configurable through hardwired connectors for each transducer array and/or by said control processor,
so that the ADC conversion takes form as one of
a) for each ADC, L of the received signals from said elements or groups of elements are in a recurring sequence connected to the ADC and converted sequentially by said ADC so that each of said signals are sampled and converted with the sample rate fₛ/L, and
b) the number of N ADCs are subdivided into groups with M ADCs in each group, where each of said group of M ADCs convert the received signal from the same said elements or groups of elements, with a delay shift between the ADCs sampling in each group of 1/Mfₛ, and the outputs of said group of M ADCs are in said digital beam forming circuits arranged to form samples of said signals with sampling rate Mfₛ,
so that the control processor for each transducer array that is coupled to the beam former, can configure the beam former to operate said array with L*N elements where the signal from each element is sampled at a frequency fₛ/L, or an ultrasound transducer array with N/M elements where the signal from each element is sampled at a frequency up to M*fₛ, all with capabilities of electronic direction steering of the beam, and without direction steering of the beam, the beam former can operate arrays with twice this number of elements by analog summation of paired element signals that are symmetric around the aperture center before digital conversion.

2. An ultrasound beam former according to claim 1, where the field programmable digital beam forming circuits are Field Programmable Gate Arrays (FPGAs).

3. An ultrasound beam former according to claim 1, where the field programmable digital beam forming circuits are made as Application Specific Integrated Circuits (ASICs).

4. An ultrasound beam former according to claim 1, where said multiplexers are programmable to select to the output free subgroups of said multiplexer inputs in a sequence, the number of inputs in said subgroups being from 1 to all of the multiplexer inputs.

5. An ultrasound beam former according to claim 4, where the number of transmit/receive circuits connected to each array element is selectable by the control processors to optimize the receiver Noise Figure and transmitter drive capabilities for the electrical impedance of the actual array elements.

6. An ultrasound beam former according to claim 1, where the sampling rate fₛ/L or Mfₛ is set for oversampling of the signal relative to the signal bandwidth, and the digitally converted signals are lowpass filtered to increase the number of bits in the signal representation with a resulting sample rate that matches the signal bandwidth.

7. An ultrasound beam former according to claim 1, where said control processor is a PC, and the PC is used for visualization of the ultrasound images and preferably also processing of the received ultrasound signal to form image parameters, like Doppler parameters, to be visualized.

8. An ultrasound beam former according to claim 1, where said delay and amplitude modifications include corrections for phase front aberrations of the ultrasound wave in heterogeneous tissues.
